(19) United States
(12) Patent Application Publication
(10) Pub. No.: US 2004/0073183 A1
(43) Pub. Date: Apr. 15, 2004

(54) Feminine hygiene perineum device

(76) Inventors: Nilda Rivera-Cruz, San Juan, PR (US); Nilsa Rivera-Cruz, San Juan, PR (US)

Correspondence Address: Nilda Rivera-Cruz, 1279 Montecarlo Avenue, Montecarlo, San Juan, PR, 00924, US

(21) Appl. No.: 10/272,066

(22) Filed: Oct. 15, 2002

(51) Int. Cl. A61F 13/15; A61F 13/20

(52) U.S. Cl. 604/385.17

(57) ABSTRACT

A Feminine Hygiene Perineum Device insertable into the perineal space of the female to prevent that the bodily fluids secreted from a user slip through the perineal space rearwardly. Said device, comprising at least an absorbent core system, having a predetermined shape, length, and width to substantially fill and comfortably fit into said perineal space, whereby preventing that the bodily fluids secreted from a user slip through the perineal space. The Feminine Hygiene Perineum Device will improve the collection of bodily fluids such as, but not limited to, menstrual fluids, urine, and vaginal discharges left unabsorbed by feminine hygiene devices.

### BACKGROUND OF THE INVENTION

The present invention relates generally to feminine hygiene devices that provide discretely a better leak protection.

Various forms and varieties of feminine hygiene devices configured for the absorption of body fluids such as menses, urine and vaginal discharges are well known. The art has offered at least three main basic types. The basic type, sanitary pads, including napkins and panty liners, are designed and developed for external wear about the pudendal region. The second type, interlabial devices, are specifically designed to be worn interlabially by female wearers. The last type are tampons, that were designed and developed for internal wear within the vaginal cavity for interruption of menstrual flow therefrom.

Sanitary pads and interlabial devices were developed for external wear about the pudendal region of the wearer. Said devices have been modified, constructing it thinner and longer, bulkier and longer at both extremes to avoid the so called accidents that could be translated into wetting and or soiling of the interior and exterior clothes.

Prior art solutions has transformed the sanitary pad thinner and longer, bulkier, with wings and with channels for a better control of fluids. These modifications has transformed the device in an uncomfortable article that does not necessarily translates into more protection. On the contrary, it turns the sanitary pad unsuitable to use with bathing suits, tight or light colored clothes because you can see the pad through the clothes.

Another prior art solution, although not with great commercial success, are interlabial devices. Interlabial devices that are not retained where inserted have an increased potential for leakage and discomfort. Interlabial devices are relatively smaller than sanitary pads and, for this reason, should capture discharged fluid directly as it exits the vaginal or urethral openings. If an interlabial device's initial position is altered, fluids can travel around it and stain the user's body or undergarments. Recent modifications has transformed the interlabial devices with tails or thongs to improve the retention of said device close to the body.

Interlabial devices are used preferably for incontinence. Once the device is wet, you dispose of it and put on a new one. During menstrual periods, you may be spending a lot of money disposing of the device because of the frequency of the wetness feeling. Menstrual periods are times when women are more sensitive mentally and physically. For this reason, users find these devices uncomfortable because, in addition to the natural discomforts of menstrual periods, you feel the bulkiness and the wetness.

In addition to the sanitary pads and interlabial devices we have the choice of tampons. The situation with tampons is its difficult insertion into the vagina and its uncomfortableness when it is not properly inserted or when women are not too wet inside. In some cultures, mothers have the apprehension with their pre adolescents and adolescents girls and their virginity. Other women find them uncomfortable physically or physiologically. Above all these inconvenients, prevail the fear of tampon users to the illness known as toxic shock syndrome. Although facing all these problems, women who cannot afford accidents related to the menstrual period, does not had any other alternative to tampons.

Various types of feminine hygiene devices have been provided in the art.

One prior art solution, Knox-Sigh, U.S. Pat. No. 5,520,675 entitled Feminine hygiene pad discloses a pad including a tail that extends rearwardly from a main portion of the pad to be received within the buttocks of a wearer. The tail insures that there will be no leakage flow rearwardly into the buttocks of the wearer, as has typically occurred with prior art pads.

The limitation of this Feminine hygiene pad resides in the fact that in horizontal or sitting positions the portion of the device that will first receive the fluids will be the tail. This means that, even when the pad may be almost dry, said tail will be soiled. An extremely soiled tail will bring the same protection as if wearing no tail at all. Is common knowledge that during menstrual periods, due to a myriad of reasons like pain and painkillers that affect the stomach, some women suffer from frequent bowel movements. If the user has a physical need to go to the bathroom, she will have to dislodge said tail and insert it again after finishing, touching the soiled portions of said device. If the user does not want to touch the soiled portion, she will have to use a new one.

Hershberger, et al, U.S. Pat. No. 6,045,544 entitled Shaped absorbent interlabial device discloses an absorbent interlabial device that consistently blocks the urethra so that it provides protection against incontinence, menstrual discharges, and discharges of bodily exudates throughout a great range of wearer motions.

This Shaped absorbent interlabial device overlooked the fact that, at the beginning of the menstrual period, some women have heavy flow and will have to change said device too frequently, spending a lot of money. Another limitation of this device is that the perineal space is not blocked and accidents can still occur.

Glasgow, et al, U.S. patent application No. 20,020,082,577 entitled Sanitary napkin with intergluteal strip discloses a sanitary napkin having a front flap that adhesively attaches to the user's undergarment and a tail strip that extends rearwardly to reside in the user's intergluteal crevice. The pad fits more snugly against the body of the user. Glasgow claims further that, because the strip provides improved body contact, similar protection is achieved with a smaller pad, thus providing a discretion.

Once again, like in the Patent issued to Knox-Sigh previously described, the limitation here resides, also, in the fact that the portion of the device that will first receive the fluids will be the intergluteal strip. Even when the napkin may be almost dry, said intergluteal strip will be soiled. In this case, when a user has the physical need to go to the bathroom, she will need to contort herself to dislodge the napkin from between the labia. The user will have to hold the device away to prevent that the napkin make contact with the water of the toilet, and make efforts to keep the portion of the intergluteal strip and the area with adhesive stay in place. To prevent all this, the user will have to change with a new device.

All beforementioned prior art devices overlooked the fact that menstrual fluids also comes with tissues from the uterus, making these devices uncomfortable due to its closeness to the body.

While these units may be suitable for the particular purpose to which they address, they would not be suitable for the purposes of the present invention as heretofore described.

The present invention has no prior art.

### BRIEF SUMMARY OF THE INVENTION

The primary object of the present invention, when used in conjunction with other feminine hygiene devices, is to block the perineal space from the bodily fluids secreted from a user. The blocking of the perineal space prevents that said fluids slip through the perineal space rearwardly, overcoming the shortcomings or accidents of the beforementioned devices.

As used herein the term perineal space refers to the genital area, and in this area, the region between the vaginal orifice and the anus orifice in the female body. The term accidents refers to as when a female wearer wet and or soil her interior and or exterior clothes, and wet and or soil the place were she was seated, and wet and or soil the bed and or blankets with bodily fluids.

The principal problem regarding accidents, during menstrual periods, lays in the fact that feminine hygiene devices creators has not realized that menstrual fluid is not completely liquid. Menstrual fluid is a part liquid and part dense, semi-gelatin or viscous substance sometimes accompanied by tissues that cannot be absorbed immediately by feminine hygiene devices. When a woman is seated or in an horizontal position, menstrual fluids tend to slips through the perineal space traveling rearwardly, leaving feminine hygiene devices behind and soiling the interior clothes and whatever comes in contact with the fluid. No matter how long, bulk or absorbent the feminine hygiene device is. Tampons, in the other hand, keep all fluids, and tissues that body secretes, inside the vagina.

The greatest incidence of accidents during menstrual periods, while using sanitary pads or interlabial devices, are when wearers are seated for long periods of time like when at work, movies, restaurants, driving and in horizontal positions like while resting or sleeping. In the sitting position, the viscous fluid that is not immediately absorbed by the sanitary pad slips through the perineal space. This fluid travels between the buttocks rearwardly where the body is not receiving the weight pressure. This place is usually in the back of the body far away from the sanitary pad. In the sleeping or horizontal position the gravity force send the fluids inevitably through the perineal space rearwardly. Almost the same occurs during incontinence situations.

Therefore, a need exists for a Feminine Hygiene Perineum Device to be used with other available fluid management products, such as sanitary pads, interlabial devices and the like, and to be placed into the perineal space, with a predetermined shape and size that consistently and substantially blocks the perineal space to prevent that bodily fluids secreted from a user slips through the perineal space rearwardly.

A need also exists for a Feminine Hygiene Perineum Device that, because of its size, could be reliably expelled when the wearer urinates, may be disposed of without difficulty and is capable of being flushed through any normal plumbing system.

A need also exists for a Feminine Hygiene Perineum Device that is small in size and that can be wore with any kind of clothes without fear that the device could be easily seen through the clothes, providing protection against menstrual discharges, incontinence, and discharges the body exudates throughout a great range of wearer motions.

It is, therefore, an object of the present invention to provide a Feminine Hygiene Perineum Device to be placed into the perineal space, with a predetermined shape and size that consistently and substantially blocks the perineal space, providing protection against accidents throughout a great range of wearer motions and positions preventing said fluids slips through the perineal space rearwardly.

Another object of the present invention is to provide a Feminine Hygiene Perineum Device that is small in size, to be positioned into the perineal space and that will be reliably expelled when the wearer urinates so that the wearer does not have to touch the soiled product.

To the accomplishment of the above and related objects, this invention may be embodied in the shapes illustrated in the accompanying drawings. Attention being called to the fact, however, that the drawings are illustrative only, and that changes may be made in the specific construction and shapes illustrated and described within the scope of the appended claims. Additional objects of the present invention will appear as the description proceeds.

The Feminine Hygiene Perineum Device of the present, invention is intended to be worn independently within the perineal space. The Feminine Hygiene Perineum Device should be worn in conjunction with other available fluid management products, such as sanitary pads, interlabial devices and the like. Additionally, the device may be commercially sold as part of a kit. The complementary parts of such a kit might include wipes, applicators for inserting the articles, moisturizers and other fluid management products. Lastly, the article may be configured and sold in individual wrappers.

It will be understood that each of the elements described above, or two or more together may also find a useful application in other types of methods differing from the type described above.

While certain novel features of this invention have been shown and described and are pointed out in the annexed claims, it is not intended to be limited to the details above, since it will be understood that various omissions, modifications, substitutions and changes in the forms and details of the device illustrated and in its operation can be made by those skilled in the art without departing in any way from the spirit of the present invention.

### BRIEF DESCRIPTION OF THE SEVERAL VIEWS OF THE DRAWING

FIG. 1 is a perspective side view of the Feminine Hygiene Perineum Device of the present invention comprising a main absorbent portion with shaped end.

FIG. 1A is a back view of the Feminine Hygiene Perineum Device.

FIG. 2 is a perspective side view of other alternative embodiment of the invention comprising a main absorbent portion with shaped ends in which the device is in a more flattered shape.

FIG. 2A is a back and front view of the alternative embodiment Feminine Hygiene Perineum Device.

FIGS. 3 and 4 are perspective side views of the embodiments of the invention comprising a main absorbent portion, FIG. 3 with a shaped end and FIG. 4 in a flattered shape in which the device also comprises a liquid pervious cover layer.

FIGS. 5 and 6 are perspective side views of the embodiments of the invention comprising a main absorbent portion, FIG. 3 with a shaped end, figure 4 in a flattered shape comprising a liquid pervious cover layer and a barrier layer at the end portion 5.

### DETAILED DESCRIPTION OF THE INVENTION

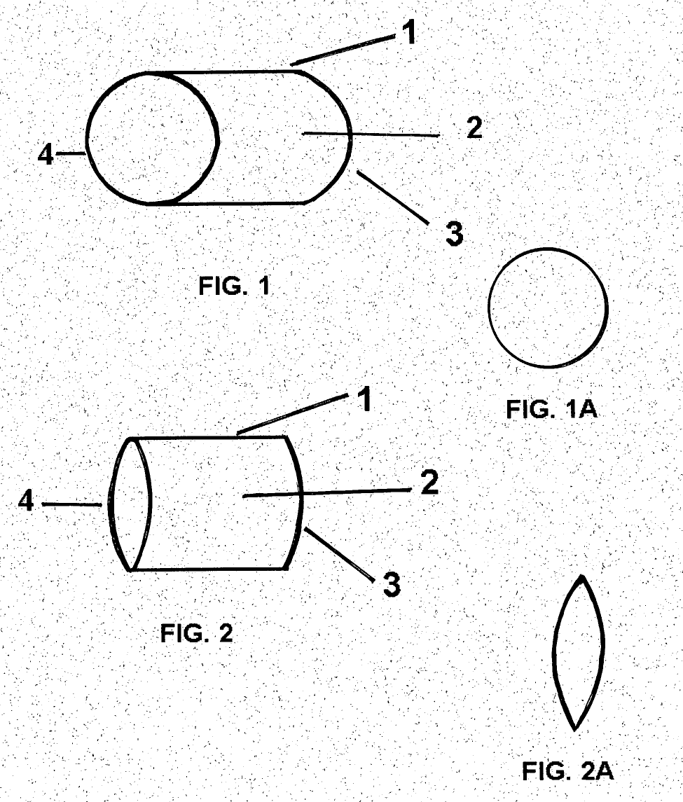

The present invention is directed to a Feminine Hygiene Perineum Device. FIGS. 1 and 2, shows the shapes of two main preferred embodiments of said device 1. The present invention, as previously explained, is not limited to the shapes having the particular configuration shown in the drawings.

As used herein the term Feminine Hygiene Perineum Device refers to a device positioned into the perineal space of a wearer to block bodily fluids secreted from a user, said Perineum Device comprising two end portions, a predetermined shape, length, and width to substantially fill and comfortably fit into said perineal space whereby preventing that the bodily fluids secreted from a user slip through the perineal space rearwardly. The Feminine Hygiene Perineum Device which has at least some absorbent components, and which is specifically configured to reside completely within the perineal space of a wearer during use. Ideally, 100% of said device 1 resides within the perineal space.

The Feminine Hygiene Perineum Device will be self retained within the perineal space of the user.

As used herein the term perineal space refers to the genital area and, in this area, the region between the vaginal orifice and the anus orifice in the female body.

As shown in FIG. 1, the Feminine Hygiene Perineum Device 1 comprises an absorbent core system or, at least, a main absorbent portion 2. The absorbent core system or main absorbent portion 2 comprises a front end portion 3 and a rear end portion 4 that is opposed to the front end portion. In use, the rear end portion 4 is positioned close to the anus orifice and the front end portion 3 is positioned close to the vaginal orifice in the genital area. Ideally, the rear end portion 4 should be covered with a barrier layer 5. The main absorbent portion 2 should be at least partially absorbent. The absorbent core system or main absorbent portion 2 may comprise non-absorbent portions, such as a liquid impervious barrier to prevent absorbed exudates from leaking out of the main absorbent portion 2.

The Feminine Hygiene Perineum Device 1 should be of a predetermined shape, length and width to substantially fill and comfortably fit into the perineal space to prevent that the fluids secreted from the body slip through the perineal space rearwardly. The Feminine Hygiene Perineum Device 1 completely blocks and intercepts the flow through the perineal space of menses, urine, and other bodily exudates from the wearer's vaginal orifice and urethra.

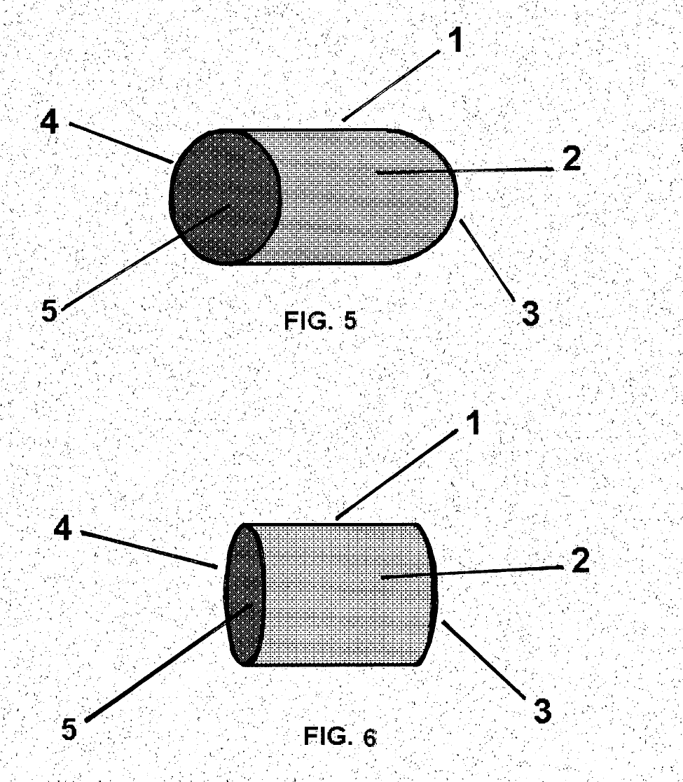

The size of the Feminine Hygiene Perineum Device 1 is also important to the comfort associated with wearing the device. In the preferred embodiment shown in FIGS. 1 through 6, the main absorbent portion 2 of the Feminine Hygiene Perineum Device 1 preferably has a length as measured along between 1 and 5 cm. The width of the main absorbent portion 2 of the Feminine Hygiene Perineum Device 1, measured along is preferably between 1 and 4 cm. If desired, a user can use a system of Feminine Hygiene Perineum Devices of varying lengths.

The Feminine Hygiene Perineum Device 1 is preferably provided with sufficient absorbency to absorb, retain and block the perineal space from the exudates discharged from the wearer's body. The capacity of the product, however, is dependent at least partially upon the physical volume of the perineal device 1, particularly the main absorbent portion 2 thereof.

The main absorbent portion 2 of the embodiments shown in FIGS. 1 through 6 may comprise any suitable type of absorbent structure that is capable of absorbing and or retaining liquids such as menses and or urine. The main absorbent portion 2 may be manufactured in a wide variety of shapes (as viewed in perspective as in FIGS. 1 and 2 or as viewed from the end as in FIGS. 1A and 2A). Viewed in perspective, as in FIGS. 1 and 2, the preferred shapes for the main absorbent portion 2 is relatively short with a slightly pointed out front end. In particularly preferred embodiments, the front end 3 of the perineal device 1 have at least a cupped shape.

The main absorbent portion 2 may, likewise, be manufactured from an absorbent core system or various liquid-absorbent materials commonly used in absorbent articles like comminuted wood pulp which is generally referred to as airfelt. Examples of other suitable absorbent materials include cotton fibers or cotton lintels, creped cellulose wadding; meltblown polymers including coform; chemically stiffened, modified or cross-linked cellulosic fibers; synthetic fibers such as crimped polyester fibers; peat moss; tissue including tissue wraps and tissue laminates; absorbent foams; absorbent sponges; super absorbent polymers in fibrous and particulate form; absorbent gelling materials; or any equivalent material or combinations of materials, or mixtures of these.

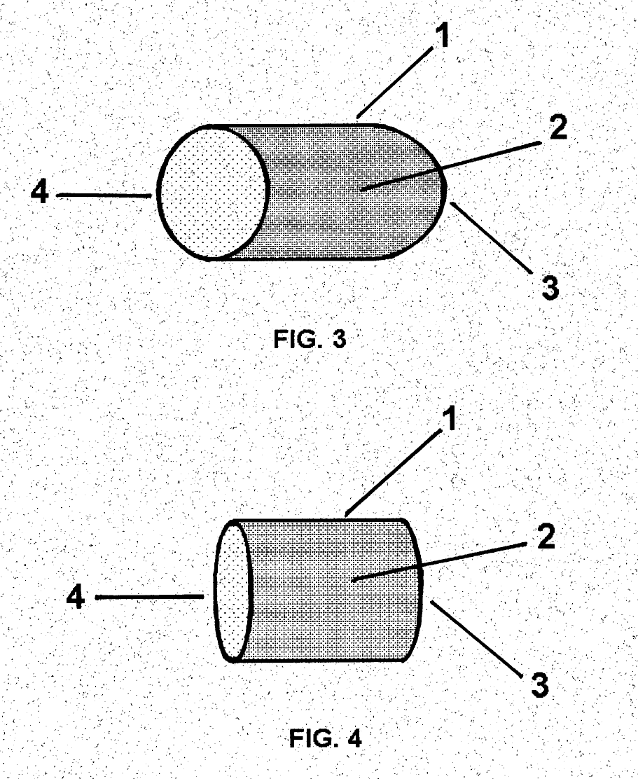

Preferred absorbent materials comprise folded tissues, cotton batts, woven materials, nonwoven webs, needle punched rayon, and thin layers of foam. The main absorbent portion 2 may comprise a single absorbent material. Alternatively, as shown in FIGS. 3 through 6, the main absorbent portion 2 may comprise a combination of materials, such as a wrapping layer 5 surrounding a central wadding comprised of an absorbent material as shown in FIGS. 3 through 6.

Providing the Feminine Hygiene Perineum Device 1 with the front end 3, having at least a cupped segment as shown in FIGS. 1, 3 and 5, endows the Feminine Hygiene Perineum Device 1 of the present invention with several advantages. Providing the device 1 with cupped front end 3 having at least a cupped segment, is particularly significant because presents more absorbent area facing toward the vaginal orifice blocking the fluid before it reaches the perineal space.

What we claim as our invention is:

1. A Feminine hygiene perineum device, to be positioned into the perineal space of a female wearer to block bodily fluids secreted from a user, said perineum device comprising at least an absorbent core system, having a predetermined shape, length, and width to substantially fill and comfortably fit into said perineal space, whereby preventing that said fluids slip through the perineal space rearwardly.

2. A Feminine Hygiene Perineum Device as recited in claim 1 comprising:
(a) at least two end portions, and
(b) an absorbent core system or a fluid absorbing material, or a combination of fluid absorbent materials thereof, between said end portions, such as plastic fibers, hydrophilic materials like hydrophilic foam and fibers, and hydrophobic materials such as cellulose fibers or wood pulp compressed into a self sustaining predetermined shape.

3. A Feminine Hygiene Perineum Device as recited in claim 2 comprising a fluid permeable cover layer such as non woven material or needle punched rayon whereby making it comfortable and safe to the wearer.

4. A Feminine Hygiene Perineum Device as recited in claim 3 comprising a barrier layer at the rear end portion whereby preventing liquids to pass through it.
